(19) United States
(12) Patent Application Publication
(10) Pub. No.: US 2008/0102450 A1
(43) Pub. Date: May 1, 2008

(54) Detecting DNA methylation patterns in genomic DNA using bisulfite-catalyzed transamination of CpGS

(76) Inventors: Michael T. Barrett, Scottsdale, AZ (US); Joel Myerson, Berkeley, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/586,884

(22) Filed: Oct. 26, 2006

(51) Int. Cl. C12Q 1/68

(52) U.S. Cl. 435/6

(57) ABSTRACT

Methods and compositions for identifying or detecting methylation patterns in a target nucleic acid are described. Cytosine residues in regions of interest are transaminated and labeled. The labeled residues are then hybridized to a microarray containing probes complementary to the region of interest, to identify the amount of methylation in the target nucleic acid.

### BACKGROUND

DNA methylation is a key process in mammalian development, believed to have a role in gene silencing, host defense against intragenomic parasites, as well as abnormal processes such as carcinogenesis, fragile site expression, etc. DNA methylation occurs through the action of the DNA methyltransferase enzyme, the predominant sequence recognition motif of which is the CpG dinucleotide. Although the CpG dinucleotide is severely underrepresented in the mammalian genome, it is found in disproportionate amounts in certain parts of the genome. It has been estimated that there are approximately 45,000 CpG islands in the human genome and 37,000 CpG islands in the mouse genome (Antequera et al., Proc. Natl. Acad. Sci. 90:11995-99 (1993)). These CpG clusters or CpG islands are present in the promoters or introns of approximately 40% of mammalian genes, and remain unmethylated in most normal cells. Increased or aberrant methylation within the CpG islands therefore can be considered a marker for abnormal or diseased states, for example, such as cancer.

The identification of targets of aberrant methylation and the characterization of patterns of methylation changes at multiple loci within a genome has potential for development of biomarkers for diseased states, the identification of novel drug targets, for monitoring response to therapy, etc. Detection of methylation patterns in a genome would also provide a tool for better understanding the biology associated with epigenetic modification of the mammalian genome.

Existing methods for detecting or identifying the methylation status of CpG sites use different technologies including Southern blotting, PCR-based amplification of genomic templates treated with methylation-specific restriction enzymes, and methods based on the use of antibodies that can bind to methylated regions. Bisulfite-catalyzed deamination, combined with DNA cloning techniques, PCR-based analyses, hybridization techniques, or microarray analysis, has also been used.

### SUMMARY

Methods for detecting DNA methylation patterns are described herein. In aspects, the methods describe transamination and labeling of unmethylated cytosine residues in a genomic sample, with the methylated cytosines in the sample remaining unlabeled. The target nucleic acids comprise fragments from a genomic sample. The target nucleic acids are hybridized to one or more microarrays comprising sequences complementary to regions of the target nucleic acids. When the signal from the target nucleic acid is compared to a signal from a reference nucleic acid, the signal provides an indication of the degree of methylation in the target nucleic acids, the location of methylation in regions of the genomic sample, or the relative degree of methylation in the target nucleic acid compared to the reference nucleic acid.

In another aspect, this disclosure describes kits for the detection of methylation patterns in a target sequence. The kits include one or more arrays containing probe sequences complementary to specific regions of a genome, or parts of a genome, along with reagents necessary for transamination and labeling or differential labeling of unmethylated cytosine residues.

### BRIEF DESCRIPTION OF THE DRAWINGS

FIG. 1 is an exemplary substrate carrying an array, such as may be used in the methods described herein.

FIG. 2 shows an enlarged view of a portion of FIG. 1 showing spots or features.

FIG. 3 is an enlarged view of a portion of the substrate of FIG. 1.

FIG. 4 shows the transamination reaction of a cytosine residue followed by a label conjugation

FIG. 5 is an illustration of the reaction mechanism for a deamination reaction catalyzed by bisulfite in a low pH environment.

FIG. 6 shows the reaction mechanism for a transamination reaction catalyzed by bisulfite at about neutral pH.

### DETAILED DESCRIPTION

Various embodiments will be described in detail with reference to the drawings, wherein like reference numerals represent like parts throughout the several views. Reference to various embodiments does not limit the scope of the claims attached hereto. Additionally, any examples set forth in this specification are not intended to be limiting and merely set forth some of the many possible embodiments for the claims.

Unless defined otherwise, all technical and scientific terms used herein have the same meaning as commonly understood by one of ordinary skill in the art. Although many methods, devices and materials similar or equivalent to those described herein can be used in the practice or testing of the methods herein, the methods, devices and materials relevant to the attached claims are now described.

All publications and patent applications in this specification are indicative of the level of ordinary skill in the art and are incorporated herein by reference in their entireties.

The term “genome” refers to all nucleic acid sequences (coding and non-coding) and elements present in or originating from a single cell or each cell type in an organism. The term genome also applies to any naturally occurring or induced variation of these sequences that may be present in a normal, mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. The methods described herein can be used to screen for particular sequences in a genome-wide (high throughput) fashion.

For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence.

The term “nucleic acid” or “polynucleotide,” as used herein, means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides. The terms “deoxyribonucleic acid” and “DNA” as used herein mean a polymer composed of deoxyribonucleotides. A “nucleotide” refers to a subunit of a nucleic acid and has a phosphate group, a 5-carbon sugar and a nitrogen-containing base. The term also refers to functional analogs of nucleotides (whether synthetic or naturally occurring), which in the polymer form (i.e. a polynucleotide) can hybridize with naturally occurring polynucleotides in a sequence-specific manner analogous to that of two naturally occurring polynucleotides. Nucleotide subunits of deoxyribonucleic acids are deoxyribonucleotides, and nucleotide subunits of ribonucleic acids are ribonucleotides.

The term “degree of methylation” refers to the amount of methylation in a first genomic sample or the ratio of methylation in each sample with respect to the other sample, when the first sample is compared with a second sample having a known amount of methylation. When the first sample is compared with a second sample having an unknown amount of methylation, the term “degree of methylation” refers to the ratio of methylation in each sample with respect to the other sample.

The term “target nucleic acid” refers to a region or fragment of interest in a genome, or genomic sample. The term also refers to a particular sequence of interest or fragment of interest in a gene. The term “gene,” as used herein refers to a nucleotide sequence along a chromosome that codes for a functional product (either RNA or the polypeptide translated from RNA). The term “reference sample” refers to a genomic sample or genomic DNA sample which is used as a reference, and may comprise reference nucleic acids. A reference sample may also refer to a defined set of synthetic oligonucleotides containing methylated and unmethylated cytosines. A reference nucleic acid is a second target nucleic acid whose signal is compared to a first target nucleic acid in order to provide an indication of the degree of methylation in a potentially methylated region of the first target nucleic acid, or the relative degree of methylation of the first target nucleic acid with respect to the second target nucleic acid. The target nucleic acids and reference nucleic acids are produced by processing (such as cleavage or fragmentation, for example) of a genomic sample (or genomic DNA sample) by various methods known to those of skill in the art. The term “genomic sample” or “genomic DNA sample” refers to the whole genome, parts of the genome, a chromosome or chromosomes, fragments of a chromosome or chromosomes, and any other genomic fragments prepared by methods known to those of skill in the art.

The term “oligonucleotide,” as used herein, generally refers to a nucleotide multimer of about 10 to 100 nucleotides in length, while a “polynucleotide” includes a nucleotide multimer having any number of nucleotides. The nucleotide multimer can contain naturally occurring as well as modified bases, or non-naturally occurring bases, including bases that can reduce the secondary structure of the oligonucleotide, such as unstructured nucleic acid (UNA) oligonucleotides, as described in U.S. Patent Application No. 20050233340, and references cited therein. An “oligonucleotide probe” refers to a moiety made of an oligonucleotide or polynucleotide, containing a nucleic acid sequence complementary to a nucleic acid sequence present in a portion of a polynucleotide such as another oligonucleotide, or a target nucleic acid sequence, such that the probe will specifically hybridize to the target nucleotide sequence under appropriate conditions.

The term “sample,” as used herein, relates to a material or mixture of materials, typically, although not necessarily, in fluid form, containing one or more components of interest. Samples include, for example, samples containing DNA, non-reduced complexity genomic DNA, DNA rich in CpG dinucleotides, etc. The sample may be directly isolated, in which case it may contain methylated as well as unmethylated regions. The sample may be obtained by amplification of a directly isolated sample, in which case the process of amplification results in oligonulceotides that no longer have methylated and non-methylated regions. Such samples may be derived from natural biological sources such as cells or tissues. A “biological fluid” includes, but is not limited to, blood, plasma, serum, saliva, cerebrospinal fluid, amniotic fluid, etc., as well as fluid collected from cell culture medium, etc.

The term “array” encompasses the term “microarray” and refers to an ordered array presented for binding to nucleic acids and the like. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

A chemical “array”, or “microarray”, unless a contrary intention appears, includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). The terms “array” and “microarray” are used interchangeably herein.

In those embodiments where an array includes two or more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one that is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this disclosure, the scan region includes the entire area of the slide scanned in each pass of the lens, between the first feature of interest, and the last feature of interest, even if there are intervening areas that lack features of interest.

An “array layout” refers to one or more characteristics of the features, such as feature positioning on the substrate, one or more feature dimensions, and an indication of a moiety at a given location. “Hybridizing” and “binding,” with respect to polynucleotides, are used interchangeably.

The term “substrate,” as used herein, refers to a surface upon which marker molecules or probes, e.g., an array, may be adhered. Glass slides are the most common substrate for biochips, although fused silica, silicon, plastic, flexible web and other materials are also suitable.

The terms “hybridizing specifically to” and “specific hybridization” and “selectively hybridize to,” as used herein, refer to the binding, duplexing, or hybridizing of a nucleic acid molecule preferentially to a particular nucleotide sequence under stringent conditions.

The term “stringent assay conditions,” as used herein, refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

In this specification and the appended claims, the singular forms “a,” “an” and “the” include plural reference unless the context clearly dictates otherwise. Unless defined otherwise, all technical and scientific terms used herein have the same meaning as commonly understood by one of ordinary skill in the art.

### Methods for Detecting Cytosine Methylation

Methods for detecting methylation patterns in a genomic sample are provided herein. In one embodiment, unmethylated cytosines in at least one target nucleic acid are transaminated, labeled and the target nucleic acid is hybridized to one or more microarrays containing probes complementary to a specific region of the target nucleic acid. Hybridization to particular sequences on the microarray coupled with the labeling of the transaminated cytosines provides for detection of the presence or absence of methylated sequences and amounts of methylation in a specific region of interest. In some embodiments, the specific region of interest is the CpG-containing region (or regions) of the target nucleic acid, and the target nucleic acids are hybridized to one or more microarrays containing probes complementary to CpG-containing regions of the target. In some embodiments, the region of interest is a potentially methylated region (or regions) that are not CpG containing regions, and the target nucleic acids are hybridized to one or more microarrays containing probes complementary to the potentially methylated regions of the target. In other embodiments, control features of the microarray can contain probes that are complementary to known non-methylated areas of the genome, or non-CpG regions of the target nucleic acid. Comparing the signal from the labeled target nucleic acids and at least one reference sample or reference nucleic acid on the one or more microarrays determines the degree of methylation in the region of interest of the genome.

The description herein provides methods for determining whether methylated sequences are present in a CpG-rich region of a target nucleic acid or target region of a genome. In embodiments, the methods are used to detect the presence of methylated CpG sequences in a region of interest, by exploiting the ability of unmethylated cytosine residues in CpGs to undergo transamination. The transaminated cytosines in at least one of the target nucleic acids are then labeled and the at least one target nucleic acid is hybridized to a microarray. Detection of the signal from the microarray helps determine the degree of methylation in the target nucleotide. In embodiments, the methods described herein can be used as a screening tool, for example to determine both areas of unknown CpG methylation in a genomic sample (i.e. location of potential methylation sites is unknown) and areas of known CpG methylation (i.e. using probes against specific regions of interest in a genome or part of a genome to determine the amount of methylation in a specific region of interest known to be a site of potential methylation). It should be recognized that the methods herein are applicable to determination of methylation in non-CpG regions of a target nucleic acid or target region of a genome, because methylation can also occur in non-CpG regions.

### Transaminating the Unmethylated Cytosine in a Target Nucleic Acid Sequence

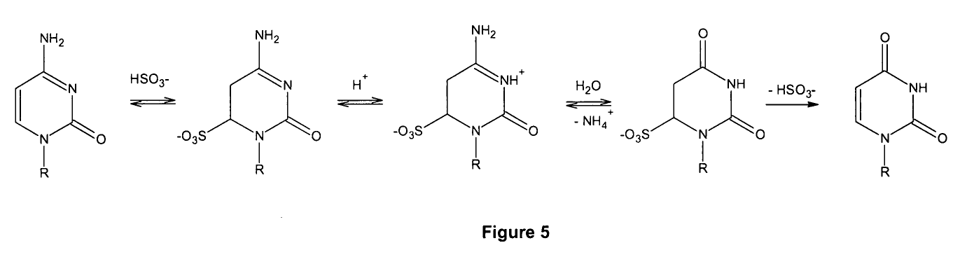

The methods described herein differentiate between unmethylated and methylated cytosine residues in target nucleic acids, using bisulfite-mediated transmination. Bisulfite has been previously used in methods for determining methylation in nucleotide sequences. For example, bisulfite nucleotide sequencing, as described in Frommer et al., Proc. Natl. Acad. Sci. 89: 1827-31 (1992), relies on the ability of sodium bisulfite, at low or acidic pH, to catalyze deamination of unmethylated, but not methylated cytosine residues, converting them into uracil, as shown in FIG. 5, allowing the unmethylated cytosines to be distinguished from the methylated cytosines in a particular sequence. In contrast, the methods described herein use transamination (rather than deamination) to distinguish between unmethylated and methylated CpGs.

In embodiments, the present methods for determining methylation in a genomic sample comprise several steps. A genomic sample is processed (using standard methods known in the art) to produce fragments, i.e. segments of DNA or unstructured regions of RNA. The nucleic acid fragments formed will include the target nucleic acid sequences. The formed fragments will be between about 40 nucleotides in length to about 1000 nucleotides in length, although shorter or longer fragments can also be used. Fragments may be single-stranded or double-stranded, and methods for denaturing double-stranded fragments to make single-stranded fragments are well known in the art. In some embodiments, this fragmentation or processing step may be omitted, where the genomic sample already comprises fragments of nucleic acids.

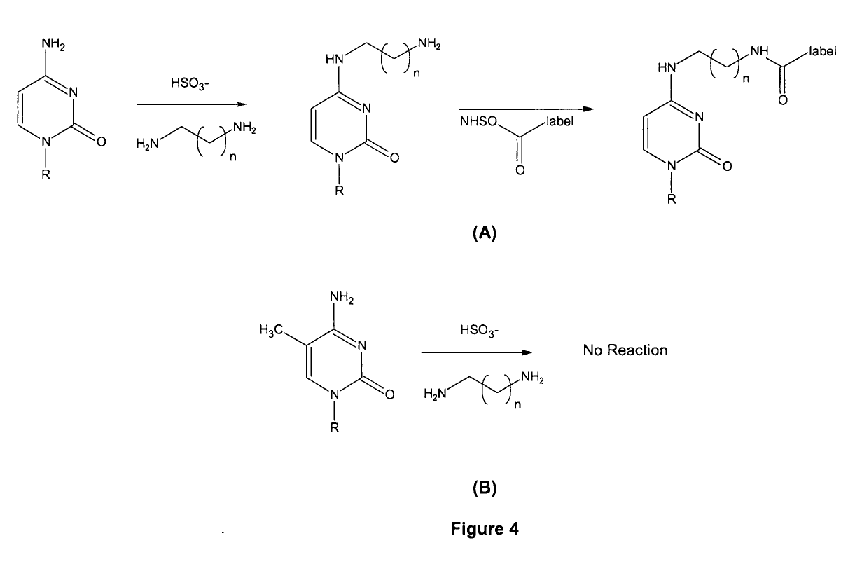

In embodiments, the genomic sample is then contacted or treated with a nucleophilic reagent, such as an amine or a hydrazide for example, in the presence of bisulfite. This results in a chemical reaction with unmethylated, but not methylated, cytosine residues. The reaction introduces side chains into the unmethylated CpGs, which can be used to label the unmethylated CpGs. For example, the side chains can be conjugated to an identifiable tag, such as, for example, a fluorophore. A schematic representation of the basic mechanism for a transamination reaction of this type is shown in FIG. 4. In an alternative embodiment, the side chain itself can contain the identifiable tag, eliminating the need for a two-step process. Microarray experiments are then performed, using arrays with probes complementary to relevant CpGs. The signal from the microarray indicates the degree of methylation of CpGs in the sample, when compared to a reference sample, a region with a known methylation pattern, or a region containing non methylated sequences.

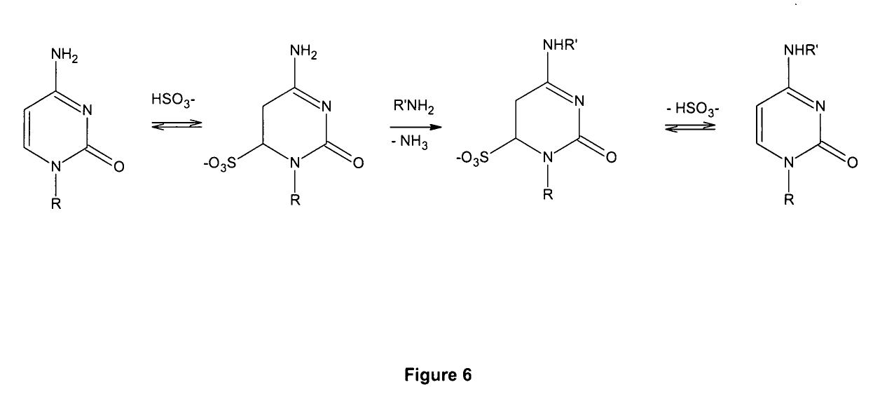

In embodiments, methods to differentiate between unmethylated and methylated CpGs using transamination are described herein. In aspects, the transamination step selectively targets at least one cytidine or deoxycytidine residue present in a sample containing 5-methyl cytidine or 5-methyl-deoxycytidine. This selectivity arises because 5-methyl cytosine, unlike cytosine, does not readily form a bisulfite adduct (as seen in FIG. 4(B)). The transamination (as shown in FIG. 6, (where R′ represents an amine), represents a predominant reaction mechanism at pH of about 5-8. At low or acidic pH, protonation occurs at the N³ position of the bisulfite adduct, and this protonated species readily undergoes deamination in the presence of water, as shown in FIG. 5. The protonation of the adduct does not occur appreciably at pH of about 7 or higher, and under such conditions the bisulfite adduct does not undergo appreciable deamination. Thus, in contrast to the deamination, which occurs at low pH, treatment with bisulfite and amine at weakly basic, neutral, or weakly acidic pH results in transamination rather than deamination of unmethylated cytosine residues. That is, at approximately neutral pH, the reaction pathway is shifted in favor of transamination and deamination is minimized or disfavored. In addition, at the low pH used for deamination, DNA degradation is known to occur. At the higher pH used during the described transamination process, there is minimal DNA degradation. Thus, by careful control of the pH conditions, unmethylated cytosine residues in CpGs can be selectively transaminated (with respect to methylated cytosine residues) without appreciable deamination occurring.

In some embodiments, the methods described herein employ various amino compounds for transamination. For example, amino compounds such as amines, hydrazides, carbazides, semicarbazides, etc. can be used for transamination. In other embodiments, highly nucleophilic amines are used, where transamination can occur without the use of bisulfite. Highly nucleophilic amines include, without limitation, 4-aminoxybutylamine, hydrazine, etc.

In some embodiments, the transamination reaction comprises treating the target nucleotide sample at a pH that promotes transamination of cytosine while minimizing deamination. In some embodiments, the pH is substantially neutral. In some embodiments, the pH is about pH 5 to about 8. In some embodiments, the pH is about 6.8 to 7.2. In some embodiments, the pH is about 7 or greater.

The transamination reaction will potentially occur with all unmethylated cytidine or deoxycytidine residues. Practically, however, the extent of such transamination is typically between about 1 and about 30 mole %, but may be substantially lower, or higher. In one extreme case, where all of the cytidines in a target are methylated, zero percent of the cytidines will undergo transamination.

The transamination reaction may also occur with unmethylated cytidine and deoxycytidine residues present in non-CpG regions of the target nucleic acids. Careful control of the length of the target nucleic acids improves the ability of the assay to distinguish targets that contain methylated and non-methylated cytosines. In embodiments, the target nucleic acids will be between 40 bases and 1000 bases in length, although targets of both longer and shorter lengths can be used.

The length of the target nucleic acid, along with the proportions of unmethylated and methylated cytosines present in the target, affect the ability of the methods to detect differences in methylation. Because the transamination reaction does not distinguish unmethylated cytosines within CpG island regions from unmethylated cytosines in non-CpG island regions, the greater the amount of non-CpG cytosine, the more difficult it is to measure differences in methylation in the CpG regions. In a genomic sample that contains both cytidine in CpG island regions and cytidine not in CpG island regions, it is desirable that the target nucleic acid contain a high proportion of cytidine in CpG islands and a low proportion of the cytidine that is not in CpG islands. One method of ensuring a high proportion of CpG islands is to fragment the genomic sample into target nucleic acids that contain a high proportion of CpG islands. The upper limit of desirable size will be a reflection of the density of CpG islands in a target nucleic acid. For a low density of CpG islands in a target nucleic acid, a target nucleic acid of 40-100 nucleotides in length may be desirable. For a high density of CpG islands in a target nucleic acid sequence, a target nucleic acid may be as large as 100-1000 nucleotides in length, for example. Another method of obtaining a target nucleic acid that has a high proportion of CpG islands is to fragment the genomic sample into smaller fragments, for example, fragments of approximately 40-100 nucleotides. This method is more likely to create at least some target nucleic acids that have a high proportion of CpG.

Other factors that can enhance the ability of the assay to distinguish methylated cytosines in a target nucleic acid include controlling the degree of transamination, by changing the time and/or temperature of the transamination reaction for example, and limiting the target nucleic acids to a region rich in CpGs.

### Labeling the Transaminated Cytosines

In some embodiments, the transaminated cytosines in the target nucleic acids are labeled. In some embodiments, at least one reference nucleic acid is labeled for comparison with the transaminated cytosines in the target nucleic acids. When the target nucleic acid is labeled and a reference nucleic acid is labeled, they may be labeled with the same or different label. When the target nucleic acid and the reference sequence are labeled with the same label, they are hybridized to separate microarrays.

In some embodiments, the transamination reaction can be utilized to introduce the label. Transamination of cytosine provides a method for introducing a label into nucleotide sequences containing cytidine or deoxycytidine. The reaction (as shown in FIG. 4 or FIG. 6) introduces side chains at the N⁴ position of the unmethylated cytidine or deoxycytidine. These side chains serve as sites for labeling. Particularly useful are side chains containing such reactive functionalities as amine groups or carboxylic acid groups. The side chains can be used to attach a variety of different nonradioactive labels, including, without limitation, dyes, fluorescent dyes, fluorophores, chromophores, Cy-3, Cy-5, as well labels such as biotin or dioxigenin, that can be further labeled in secondary reactions with molecules or macromolecules well known in the state of the art, such as fluorescently labeled avidin or anti-dioxigenin, anti-biotin, phycoerythrin, nanoparticles, entities which can provide further signal amplification such as, for example, enzymes like horseradish peroxidase, or oligonucleotide primers for rolling circle amplification.

In an aspect, the label identifies the unmethylated cytosine residues in a CpG-rich region of the target nucleic acid sequence. In another aspect, the relative intensity of label (such as a fluorophore or dye tag, for example) on the unmethylated cytosine residue can be used as an indication of the amount of unmethylated cytosine in a CpG-rich region of the target nucleic acid sequence, when compared to a reference sample, for example.

In embodiments, the methods described herein use a two-step process for labeling the unmethylated cytidine or deoxycytidine residues. In an aspect, the transamination reaction with a diamine is performed first, following by conjugation of the resulting amino compound with the appropriate label compound. In another aspect, the transamination reaction with an amino carboxylic acid is performed first, followed by conjugation of the resulting carboxy compound with the appropriate label compound. In another aspect, the transamination and labeling can be combined in a single step process, incorporating a biotin label for example, by performing the transamination reaction with a biotin containing amino compound.

In embodiments, the methods described herein are used in a two-color microarray analysis. In the two-color assay, a first sample is labeled with a first fluorescent label, and a second sample is labeled with a second fluorescent label that is distinguishable from the first label (i.e. the two samples are differentially labeled).

In some embodiments, when a one-color assay is utilized, the CpG island containing target nucleotide sequence and at least one reference nucleic acid are labeled with the same label. When the one-color method is utilized, the reference sample and the CpG island containing target nucleotide sequence are each applied to separate microarrays and then compared.

### Hybridizing the Labeled Target Nucleotide Sequence to Microarrays

In embodiments, the labeled target nucleic acids are hybridized to one or more microarrays, including CGH arrays, CpG island arrays and the like. In one embodiment, the microarray may comprise probes that can detect overlapping sequence regions of the target nucleic acid in order to determine the location of the unmethylated cytosines in the genomic sample. In another embodiment, the microarray comprises probes that are complementary to a specific region of the target nucleic acid, such as a CpG-containing region, for example. In another embodiment, the microarray comprises probes that are complementary to regions near or adjacent to CpG containing regions. Methods of designing arrays and probes have been described in U.S. Pat. Nos. 6,242,266, 6,232,072, 6,180,351, 6,171,797, and U.S. Pat. No. 6,323,043, and the references cited therein, and methods for designing probes that can hybridize to overlapping regions have been described in U.S. Patent Publication No. 20060110744, and references cited therein.

In embodiments, labeled target nucleic acids and reference samples are hybridized to one or more microarrays. The reference sample may be a nucleic acid with a known degree of methylation, or a nucleic acid with an unknown degree of methylation, The reference sample may be a nucleic acid that is from the same genomic sample as the target nucleic acid sequence. The reference nucleic acid may be either an internal reference standard or an external reference standard.

In some embodiments, a labeled target nucleic acid may be hybridized to the same array as a reference sample. In other embodiments, the labeled target nucleic acid sequences may be hybridized to separate arrays comprising the same probes to which the target nucleic acids hybridize. The separate arrays may be of the same type, where same type means that a first array and a second array have substantially the same probes, or the second array may have additional features not present on the first array. In an embodiment, only one sample is labeled, and known non-methylated regions are used as an internal standard. Information about the location and degree of methylation can be obtained by comparing hybridization to probes complementary to specific regions of the target nucleic acid (such as the CpG region) with probes that are complementary to known non-methylated regions of the genome. In another embodiment, two samples are labeled, one sample being a reference sample, and the two samples are hybridized to separate arrays. In yet another embodiment, a reference sample is obtained by splitting a single sample, and treating part of the sample to generate an unmethylated reference sample. In embodiments, two samples are differentially labeled and hybridized to the same microarray. Differential labeling occurs where a first sample is labeled with a first label, and a second sample (such as a reference sample) is labeled with a second label, each label being distinguishable from the other (such as by color, for example). Comparing the signals from the differentially labeled samples provides information regarding the extent or amount of methylation in the regions of interest in each sample.

In embodiments, the methods described herein are used in a two-color microarray analysis. In the two-color assay, a first sample is labeled with a first fluorescent label, and a second sample is labeled with a second fluorescent label that is distinguishable from the first label (i.e. the two samples are differentially labeled). In some embodiments, the second sample is a reference sample. In embodiments, the reference sample can have either a known degree of methylation, or an unknown degree of methylation. After labeling, the two samples are simultaneously hybridized on a single microarray. By comparing the relative ratios of signal intensities of the two samples at the two appropriate wavelengths, the relative amounts of methylation between the two samples can be obtained. Each individual probe site will reflect the amount of label incorporated in the unmethylated cytosines, which will reflect the amount of unmethylated cytosines present in the hybridized targets.

The methods described herein can be used in one-color microarray analysis, with either internal reference standards, or external reference standards. When an internal standard is used, one or more samples are hybridized to identical arrays, and appropriate probes that hybridize to known non-methylated regions (appropriately matched for hybridization stringency) can be used as internal standards. Comparison of the arrays, together with the internal standards, can give an indication of the locations and relative amounts of methylation within samples and between samples. When an external standard is used, a first sample is hybridized to a first array, while a second sample (i.e. the external reference standard) with a known degree of methylation and known methylation pattern is hybridized to a second array of the same type. Comparison of the two arrays, together with knowledge as to the methylation of the external standard, provides an indication of the locations and relative amounts of methylation in the first sample.

In embodiments, the methods herein can be used in conjunction with microarray analyses using unmethylated reference standards, i.e. reference samples that do not contain any methylation sites. Such samples can be prepared by polymerase extension (and subsequent amplification, if desired) of genomic template DNA using a variety of polymerase enzymes. The resulting DNA will not contain methylated cytidines, and can be separated from the original methylated DNA (using biotinylated primers, for example), or sufficiently diluted out by the amplification process. Alternatively, an unmethylated reference sample can be obtained using enzymes such as DNA demethylase. Use of an unmethylated reference is particularly advantageous in the case where the unknown genomic sample itself is used to prepare the unmethylated reference. The sample is split into two parts, one part of which is labeled by transamination, and the other part of which is turned into an unmethylated reference by the methods described.

The methods described herein can be the basis of array-based applications for analyzing DNA methylation. In embodiments, the transaminated and labeled unmethylated CpGs are used in a microarray assay, screening nucleotide target sequences for the presence or absence of methylation in a region of interest. In an aspect, the microarray assays can be used to detect aberrant methylation in a chromosome-wide analysis. In another aspect, the assays can be used for promoter-specific analysis to identify sites of methylation.

In embodiments, screening of multiple CpG sites up to an entire genome can be conducted in a single assay. For example, if a tiling array is used, CpG methylation in any region of the genome can be studied, without the need to define specific probes. A tiling array is type of microarray where probes are not designed to target known genomic regions, e.g., genes or portions thereof, such as coding sequences, promoters, etc. Rather, probes are simply laid down at regular intervals along the length of the genome. Tiling arrays include arrays of overlapping oligonucleotides that represent an entire genomic region of interest, such as a chromosome, for example. In one aspect, because specific probes complementary to coding sequences are not required, methylation patterns in any region of the genome can be analyzed in a single assay. In another aspect, methylation patterns in many regions of the genome can be analyzed simultaneously in a single microarray experiment.

In embodiments, the methods described herein enable high throughput screening of methylation patterns, by elimination of error-prone, DNA-degrading and labor-intensive experimental steps. The use of transamination rather than deamination eliminates problems associated with degradation of DNA, and incomplete or biased conversion of cytosine to uracil. In an aspect, transamination followed by microarray analysis eliminates the need for labor intensive procedures such as cloning and sequencing of individual DNA targets to determine methylation patterns on a gene-by-gene basis, as in traditional bisulfite nucleotide sequencing. Direct labeling via transamination of unmethylated CpGs eliminates the need to use PCR-based approaches, where detection is limited to CpGs present in the recognition sites of the PCR primer (usually only about 20 to 30 nucleotides). PCR-related artifacts are also eliminated by using the methods described herein. In an aspect, the methods described herein do not rely on time- and labor-intensive procedures for bisulfite conversion, restriction digestion, PCR-based amplification, etc, of existing microarray-based methods for genome-wide analysis of methylation. In another aspect, CpG-containing sequences used in the present methods are not amplified (by enzymatic methods, for example) prior to labeling and hybridization to the microarray.

In some embodiments of the methods described herein, at least one reference sample or reference nucleic acid is utilized. The reference sample may be present on the array (internal) or may be applied to one or more arrays (external).

In some embodiments, the reference sample is obtained by splitting a single sample and treating part of the sample to generate a sample with unmethylated reference nucleic acids. The unmethylated cytosine in the reference nucleic acid or set of nucleic acids can be transaminated and labeled in accord with the methods described herein.

If the reference nucleic acid is internal, it comprises at least one oligonucleotide containing a known amount of unmethylated cytosines. Unmethylated cytosines in the reference nucleic acid are labeled with a detectable label. The amount of unmethylated cytosines in a reference nucleic acid is not critical, as long as the amount of unmethylated cytosines is known. At minimum, when the reference nucleic acid is internal, it comprises at least one oligonucleotide containing at least one unmethylated cytosine.

In some embodiments, the reference is a set of reference nucleic acids, each reference nucleic acid having a sequence that hybridizes to a microarray and has a different amount of labeled unmethylated cytosine. The amount of unmethylated cytosines in a reference nucleic acid is not critical, as long as the amount of unmethylated cytosines is known.

In some embodiments, a set of reference nucleic acids are designed to have about 10 to 100 nucleotides and each having a different amount of labeled unmethylated cytosines. For example, a set of reference sequences can include reference sequences with 1, 2, 3, 4, 5, 6, 7, 8, 9, 10, etc. and up to 30 labeled unmethylated cytosines. The labeled unmethylated cytosines in such a designed sequence can be transaminated or labeled in accord with the methods described herein.

In some embodiments, the set of reference sequences are hybridized to the array. In the situation where the reference sequences are to be hybridized to the array, each reference sequences can be designed to comprise a portion that is complementary to a probe on the array and a portion that has varying degrees of unmethylated cytosine.

### Arrays for Detecting Methylation

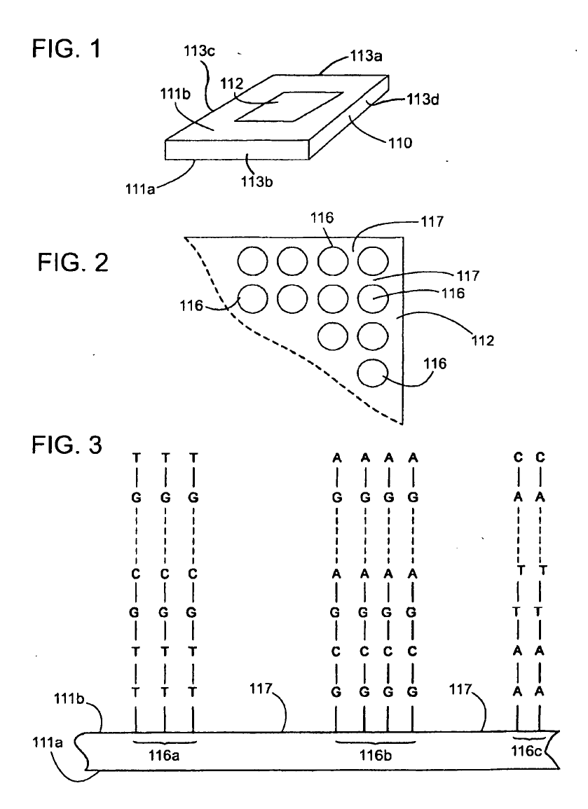

The methylated and unmethylated CpG targets in a sample can be probed using oligonucleotides with sequences that are complementary to the CpG-rich regions of the target nucleotide sequence. Alternatively probes can consist of oligonucleotide sequences complementary to non-CpG regions that are adjacent or close to the CpG region of interest. The oligonucleotide probes can contain naturally occurring as well as modified bases, or non-naturally occurring bases, including bases that can reduce the secondary structure of the oligonucleotide, such as unstructured nucleic acid (UNA) oligonucleotides, as described in U.S. Patent Application No. 20050233340 and the references cited therein, incorporated herein by reference. In one embodiment, the complementary sequences are immobilized onto a glass slide or microchip to form a DNA array or microarray. An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape.

As mentioned above, array 112 contains multiple spots or features 116 of biopolymers, e.g., in the form of polynucleotides. All of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In an embodiment, the isolated DNA fragments are then hybridized to the microarray under stringent assay conditions. Stringent assay conditions as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions. A stringent hybridization and stringent hybridization wash conditions in the context of nucleic acid hybridization (e.g., as in array, Southern or Northern hybridizations) are sequence dependent, and are different under different experimental parameters.

Stringent hybridization conditions that can be used to identify nucleic acids can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. A specific example of stringent assay conditions is rotating hybridization at 65° C. in a salt based hybridization buffer with a total monovalent cation concentration of 1.5M (e.g., as described in U.S. patent application Ser. No. 09/655,482, filed on Sep. 5, 2000, and incorporated herein by reference), followed by washes of 0.5×SSC and 0.1×SSC at room temperature. Those of ordinary skill will readily recognize that alternative but comparable hybridization and wash conditions can be utilized to provide conditions of similar stringency.

In certain embodiments, the stringency of the wash conditions sets forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions. Other stringent hybridization conditions are known in the art and may also be employed, as appropriate.

The DNA arrays described herein are arrays of nucleic acids, including oligonucleotides, polynucleotides, DNAs, RNAs, synthetic mimetics thereof, and the like. Specifically, the arrays contain spots or features in the form of oligonucleotides corresponding to specific probe sequences. The subject arrays include at least two distinct nucleic acids that differ by monomeric sequence immobilized on, e.g., covalently to, different and known locations on the substrate surface. In an embodiment, the arrays contain spots corresponding to genomic DNA sequences. In certain embodiments, each distinct nucleic acid sequence of the array is typically present as a composition of multiple copies of the polymer on the substrate surface, e.g., as a spot on the surface of the substrate. The number of distinct nucleic acid or oligonucleotide sequences, or spots or similar structures present on the array may vary, but is generally at least 2, usually at least 5 and more usually at least 10, where the number of different spots on the array may be as a high as 50, 100, 500, 1000, 10,000 or higher, depending on the intended use of the array. The spots of distinct oligonucleotide sequences present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the oligonucleotide sequences are not arranged in the form of distinct spots, but may be positioned on the surface such that there is substantially no space separating one polymer sequence/feature from another.

Arrays can be fabricated using drop deposition from pulsejets of either polynucleotide precursor units (such as monomers) in the case of in situ fabrication, or the previously obtained polynucleotide. In an embodiment, the arrays are fabricated using oligonucleotides with sequences complementary to genomic DNA. In another embodiment, separate arrays are fabricated, containing probes for genomic DNA. Methods for array fabrication are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can also be used for array fabrication.

In embodiments, the microarray is a tiling array, wherein the probes on the microarray can be overlapping as described herein to allow identification of the location of unmethylated cytosines in the genomic sample. The methods as described herein can be utilized with a plurality of target nucleic acids.

In some embodiments, the signal from the labeled CpG containing target polynucleotide is compared to the signal from at least one sample on one or more microarrays.

In some embodiments, the reference sample may include a plurality of reference nucleic acids each with a known amount of methylated cytosines and each having a different amount of unmethylated cytosines. In some embodiments, the set of reference nucleic acids is hybridized to a separate array of the same type. The signals from each of the reference nucleic acids is utilized to provide a standard curve. The signal of the CpG island nucleic acid can then be compared to the standard curve and the amount of unmethylated cytosines in the target nucleic acid can be determined.

When a one-color array is used, the target nucleic acid is labeled and hybridized to a microarray. In some embodiments, at least one reference is also labeled with the same label and hybridized to a separate microarray of the same type. The signal intensities from each microarray are compared. If the amount of signal from the target nucleic acid is greater than the reference nucleic acid, the amount of unmethylated cytosines in the target nucleic acid is greater than that of the reference nucleic acid.

When a two-color microarray is utilized, at least one containing target nucleic acid is labeled with one type of label, and one or more reference nucleic acids are labeled with a different label. In some embodiments, both the target nucleic acids and one or more of the reference nucleic acids are hybridized simultaneously to the same microarray. The signal intensity of the target nucleic acids can then be compared to the signal intensity of one or more reference nucleic acids to determine the amount or relative amount of unmethylated cytosines in the target nucleic acids.

Methods for measuring signals from microarrays and analyzing the signal intensity are known to those of skill in the art and are also available commercially.

### Kits for Detecting Methylation

The methods described herein can be used in kits for the identification or detection of methylated or unmethylated cytosine in target nucleic acids. In embodiments, the kits contain at least one suitably packaged microarray with spots corresponding to probes for the CpG rich regions of a genomic sample. In embodiments, the array is a tiling array, wherein oligonucleotide probes with sequences complementary to part or all of the entire genome are placed at regular intervals on the array substrate.

In embodiments, the kits described herein contain reagents necessary for the transamination of unmethylated cytosines, such as diaminoethane, NaHSO₃, and required buffers, for example. The kits also include reagents for the labeling or differential labeling of transaminated unmethylated cytosines in target nucleic acids containing methylated or unmethylated cytosines, such as a one or more fluorescent dyes, or biotin, for example. The reagents for labeling can be in an activated form for direct reaction with a functionality on the side chain incorporated during the transamination reaction. For example, the label can contain an N-hydroxysuccinimide ester for reaction with an amino containing side chain. The reagents for labeling can also be supplied in a non-activated form, together with an appropriate reagent for activation. For example, the label can contain a carboxylic acid that can be conjugated to an amino containing side chain in the presence of a carbodiimide reagent.

In some embodiments, the kits may include at least one or a set of reference nucleic acids, each with a known amount of labeled methylated cytosines as described previously. These reference nucleic acids can be hybridized to a separate microarray of the same type as the target nucleic acids. In some embodiments, at least one or a set of reference nucleic acids each with a different amount of labeled unmethylated cytosines may be attached to the array.

In embodiments, the kit may also contain means for fragmentation of DNA, purification of genomic DNA, purification of fragmented DNA, purification of transaminated DNA, or purification of labeled DNA. In embodiments, the kit may also contain instructions providing information on use of the microarray to detect the presence of methylated or unmethylated CpGs.

### EXAMPLES

The following examples are provided by way of illustration only, and are not intended to limit the claims.

### Sample Preparation

In the methods described herein, the target nucleic acids are prepared from a genomic sample or genomic DNA, which must be cleaved or fragmented into targets of an appropriate size. The desirable target size will vary, but typical fragments of interest will be between 40 bases and 1000 bases in length, although targets of both longer and shorter lengths can be used.

Fragmentation is typically performed prior to the transamination and conjugation steps, although it can be done after the transamination step or after the conjugation step. Cleavage and fragmentation may be achieved using any convenient protocol, including but not limited to, mechanical protocols, e.g., sonication and shearing, etc., chemical protocols, e.g., cleavage by tris(3-hydroxy-1,2,3-benzotriazine-4(3H)one]-iron(III) and other chemical agents, and enzymatic protocols, e.g., digestion by DNAses, a restriction enzyme or the like. These methods are well known in the art. For example, the genomic sample may optionally be contacted, under suitable conditions, with one or more restriction endonucleases that recognize cleavage sites that generally lie outside of CpG islands (i.e. CpG-rich regions). This contacting step cleaves the DNA in the extract into fragments in which CpG islands, methylated or unmethylated, are intact. The restriction enzymes such as AluI, RsaI, MseI, Tsp509I, NlaIII and BfaI, for example, are used for cleavage. A person of skill in the art will recognize that many other restriction enzymes can be used for this cleavage step. In some cases it may be desirable to treat the genomic DNA with enzymes that recognize cleavage sites within the CpG islands, such as BstUI, SmaI, SacII, EagI, MspI, HpaII, HhaI and BssHII

### Transamination

For transamination of unmethylated CpG-containing target nucleic acid sequences, a minor fraction of the total deoxycytidine bases that are contained in the starting genomic DNA or genomic sample are transaminated. The extent of such transamination is typically between about 1 and about 30 mole %, but may be substantially lower, particularly in targets that contain a very large proportion of methylated cytidine. In extreme cases, where all of the cytidines in a target are methylated, zero percent of the cytidines will undergo transamination.

Although not limiting the present description, typical conditions for transamination are described in U.S. Pat. No. 6,569,626, the disclosure which is incorporated herein by reference in its entirety. Briefly, fragmented DNA is denatured by boiling, and then chilled. Transamination is initiated by adding a pH 7.0 buffer that is approximately 1M in bisulfite and approximately 3M in a diamine, such as ethylene diamine, for example. The length of time the reaction proceeds will determine the degree of transamination achieved. Typical reaction times range from a few hours to a few days.

### Dye Conjugation

To conjugate the transaminated targets with appropriate dye labels, standard conjugation techniques known in the art are used. For example, N-hydroxy-succinimide esters of dyes such as cyanine-3 (Cy-3) or cyanine-5 (Cy-5) are readily coupled to a transaminated ethylene diamine linker at pH 8. The conjugation can be driven to completion by using an excess of dye, or the transaminated compound can be partially labeled by using a lower concentration of dye, or stopping the reaction before completion.

### Array Hybridization

Prior to hybridization to the arrays, the hybridization mixture is typically denatured at 100° C. for 1.5 minutes and incubated at 37° C. for 30 minutes. The sample is applied to the microarray and hybridization is allowed to proceed under temperature conditions optimized for the hybridization buffer. For example, for a buffer solution with a monovalent cation concentration of 1.5M, the optimal hybridization condition is typically about 14-40 hours at 65° C. The hybridization step may include agitation of the immobilized targets and labeled nucleic acids, and agitation may be accomplished using any convenient protocol, e.g., shaking, rotating, spinning, and the like, as known to those of skill in the art. After washing of arrays in a series of suitable buffers, such as 0.5×SSC and 0.1×SSC, for example, the slides are dried and scanned.

### Array Scanning

Reading of the resultant hybridized array may be accomplished by illuminating the array and reading the location and intensity of resulting fluorescence at each feature of the array to detect any binding complexes on the surface of the array. For example, a scanner may be used for this purpose that is similar to the AGILENT MICROARRAY SCANNER available from Agilent Technologies, Palo Alto, Calif. Other suitable devices and methods are described in U.S. patent applications: U.S. patent application Ser. No. 09/846,125, entitled “Reading Multi-Featured Arrays” and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels), or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array.

The various embodiments described above are provided by way of illustration only and should not be construed to limit the claims. Those skilled in the art will readily recognize various modifications and changes that may be made without following the example embodiments and applications illustrated and described herein, and without departing from the true spirit and scope of the following claims.

1. A method for determining the degree and location of methylation in a genomic sample, comprising:
contacting target nucleic acids in the genomic sample with a solution comprising bisulfite and an amine to transaminate unmethylated cytosines in the target nucleic acids;
labeling the transaminated cytosine in the target nucleic acids with a label;
hybridizing the labeled target nucleic acids to one or more microarrays;
detecting a signal on the one or more microarrays from the target nucleic acids; and
comparing the signal from the labeled target nucleic acids on the one or more microarrays with a reference signal to determine the degree of methylation in the target nucleic acid.

2. The method of claim 1, wherein comparing the signal from the labeled target nucleic acids with the reference signal determines the degree of methylation and/or the location of methylation in the genomic sample.

3. The method of claim 1, wherein comparing the signal from the labeled target nucleic acids with the reference signal determines the degree of methylation in CpG-containing regions of the target nucleic acid.

4. The method of claim 1, wherein the reference signal is from at least one reference nucleic acid, said reference nucleic acid comprising one or more nucleic acids with a known degree of methylation.

5. The method of claim 1, wherein the reference signal is from at least one reference nucleic acid, said reference nucleic acid comprising one or more nucleic acids with an unknown degree of methylation.

6. The method of claim 1, wherein the reference signal is from at least one reference nucleic acid, said reference nucleic acid comprising one or more nucleic acids from the same genomic sample as the target nucleic acid.

7. The method of claim 1, wherein the reference nucleic acid comprises an external reference standard or an internal reference standard.

8. The method of claim 1, wherein the hybridizing and detecting are performed in a two-color mode or a one-color mode.

9. The method of claim 1, wherein the target nucleic acids contain no unmethylated cytosine residues such that transaminating and labeling produces target sequences with no label.

10. The method of claim 1, wherein transaminating and labeling of unmethylated cytosines take place in a single step.

11. The method of claim 1, wherein transaminating the cytosine comprises treating with bisulfite at about neutral pH.

12. The method of claim 1, wherein transaminating the cytosine comprises treating with bisulfite at pH values of about 5 to about 8.

13. The method of claim 1, wherein transaminating the cytosine comprises treating with bisulfite at pH values of about 6.8 to about 7.2.

14. The method of claim 1, wherein transaminating the cytosine comprises treating with bisulfite at a pH that promotes transamination of cytosine while minimizing deamination.

15. The method of claim 1, wherein transaminating the cytosine introduces side chains at the N⁴ position of an unmethylated cytosine.

16. The method of claim 15, wherein the side chain at the N⁴ position of an unmethylated cytosine comprise an amine.

17. The method of claim 15, wherein the side chains at the N⁴ position comprise conjugation sites for fluorescent molecules.

18. The method of claim 15, wherein the side chains at the N⁴ position comprise conjugation sites for a biotin containing molecule.

19. The method of claim 15, wherein the side chains at the N⁴ position are used to label unmethylated cytosine.

20. The method of claim 15, wherein the relative intensity of the label on the unmethylated cytosine provides an indication of the degree of methylation in the target nucleic acid, where the intensity is compared to intensity from a reference nucleic acid.

21. The method of claim 1, wherein the microarray comprises oligonucleotide probes complementary to CpG-containing regions of the genome.

22. The method of claim 1, wherein the microarray comprises a tiling array.

23. A method for distinguishing methylated and unmethylated CpG in a target nucleic acid, comprising transaminating the unmethylated cytosine.

24. The method of claim 23, wherein transaminating the unmethylated cytosine comprises contacting the sample with bisulfite and amine at about pH 6.8 to about pH 7.2.

25. A method for determining the degree and location of methylation in a genomic sample, comprising:
treating the genomic sample with a solution comprising bisulfite and an amine;
labeling the treated genomic sample with a label;
fragmenting the genomic sample to produce target nucleic acids;
hybridizing the target nucleic acids to one or more microarrays comprising probe sequences complementary to the target nucleic acid;
detecting a signal on the one or more microarrays from the target nucleic acid; and
comparing the signal from the labeled target nucleic acids on the one or more microarrays with a reference signal to determine the degree of methylation in the target nucleic acid,
wherein the fragmenting step is optional where the genomic sample comprises fragments including the target nucleic acids.

26. A kit for detecting the presence of methylated and unmethylated cytosine in a genomic sample, comprising:
reagents for transaminating unmethylated cytosine in a set of target nucleic acids and in a set of reference nucleic acids;
reagents for labeling transaminated cytosines; and
instructions for the use of the kit for identification or detection of methylated and unmethylated cytosine in a genomic sample.

27. The kit of claim 26, further comprising at least one DNA array comprising a plurality of oligonucleotides having sequences complementary to CpG-containing regions in the target nucleic acids.

28. A kit for detecting the presence of methylated and unmethylated cytosine in a genomic sample, comprising:
at least one DNA array containing a plurality of oligonucleotides with sequences complementary to CpG-containing regions of interest in target nucleic acids;
reagents for transaminating unmethylated cytosine;
reagents for labeling transaminated cytosines; and
instructions for the use of the kit for identification or detection of methylated and unmethylated cytosine in a genomic sample.
